(19) United States
(12) Patent Application Publication
(10) Pub. No.: US 2013/0023701 A1
(43) Pub. Date: Jan. 24, 2013

(54) SYSTEM AND METHOD FOR OXIDIZING METHANE

(71) Applicants: Youcai Zhao, Shanghai (CN); Dan Han, Suzhou (CN)

(72) Inventors: Youcai Zhao, Shanghai (CN); Dan Han, Suzhou (CN)

(73) Assignee: EMPIRE TECHNOLOGY DEVELOPMENT LLC, Wilmington, DE (US)

(21) Appl. No.: 13/129,102

(22) PCT Filed: May 28, 2010
(86) PCT No.: PCT/CN2010/073335

§ 371 Date: May 12, 2011

(51) Int. Cl. C07C 29/48; B01J 19/24

(52) U.S. Cl. 568/910; 422/129; 422/187

(57) ABSTRACT

The present disclosure provides a system for oxidizing methane, including a construct with at least one conduit for air access, and a layer of mineralized waste surrounding the at least one conduit. The present disclosure also provides a method for oxidizing methane, including putting the system for oxidizing methane into contact with the material to be oxidized.

### BACKGROUND

Methane is a potent greenhouse gas which is estimated to be about 23 times more effective than carbon dioxide in trapping heat. Moreover, methane is explosive at a sufficiently high concentration, and therefore constitutes a safety concern at methane-emitting sites.

Landfills are major methane-emitting sources. Methane generated from decomposition of solid waste by microbial activities and chemical reactions can build up within the solid waste and be released into the atmosphere, causing great environmental problems as well as safety concerns.

Various methods have been used to reduce methane emission at landfills, including collecting methane for energy use and oxidizing methane before its emission. However, the existing methods are limited in various aspects, such as low oxidation efficiency and requirement of expensive equipment.

### SUMMARY

In one aspect, the present disclosure provides a system for oxidizing methane, which includes a construct with at least one conduit configured for air access; and a layer of mineralized waste configured to surround the at least one conduit.

In another aspect, the present disclosure provides a method for oxidizing methane, including putting a system for oxidizing methane into contact with the material to be oxidized, in which the system includes a construct with at least one conduit configured for air access, and a layer of mineralized waste surrounding the at lease one conduit.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 shows an illustrative embodiment of the system for oxidizing methane of the present disclosure.

FIG. 2 shows an illustrative embodiment of the system for oxidizing methane of the present disclosure.

FIG. 3 shows four different illustrative embodiments of the system for oxidizing methane of the present disclosure.

FIG. 4 shows a schematic drawing of an illustrative embodiment of a system for oxidizing methane of the present disclosure.

FIG. 5 shows the results of methane conversion rates in the three mineralized waste columns.

FIG. 6 shows the results of methane conversion rates in the medium sized mineralized waste column under different inlet methane loads.

FIG. 7 shows a cross-section drawing of the experimental landfill unit with a system for oxidizing methane.

FIG. 8 shows methane concentrations in the outlet gas for the control unit and the experimental unit, respectively, during the 150-day study period.

FIG. 9 shows the reduction rate of methane emission in the experimental unit as compared with the control unit, during the 150-day study period.

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented here.

The present disclosure provides systems and methods for oxidizing methane. In one aspect, the present disclosure provides a system for oxidizing methane, including a construct with at least one conduit configured for air access and a layer of mineralized waste configured to surround the at least one conduit.

The term “mineralized waste” refers to solid waste that contains organisms capable of oxidizing methane. Such organisms are referred to as methane-oxidizing organisms. The methane-oxidizing organisms can oxidize methane to produce substances such as formaldehyde, formic acid, carbon dioxide and water.

In certain embodiments, the methane-oxidizing organisms are microorganisms including, without limitation, bacteria and archaea. In certain embodiments, the methane-oxidizing bacteria are bacteria belonging to the families of Methylococcaceae and Methylocystaceae. In certain embodiments, the Methylococcaceae family includes bacteria belonging to the genera of Methylococcus, Methylomonas, Methylomicrobium, Methylobacter, Methylocaldum, and Methylosphaera. In certain embodiments, the Methylocystaceae family includes bacteria belonging to the genera of Methylocystis, Methylosinus, and Methylopila. Illustrative examples of methane-oxidizing bacteria include Methylomonas methanica, Methylococcus capsulatus, Methylosinus trichosporium, and Methylocystis parvus.

In certain embodiments, the methane-oxidizing archaea are from the families of Methanosaetaceae, Methanosarcinaceae, and Methermicoccaceae.

Methane-oxidizing organisms may naturally grow and develop in mineralized waste under suitable conditions. Generally, methane-oxidizing organisms grow in solid waste when there is accumulation of methane in the solid waste. Methane may accumulate in solid waste as the waste materials decompose and decay over time. Solid waste containing methane-oxidizing organisms becomes mineralized waste and may be used in the system for oxidizing methane of the present disclosure. In certain embodiments, the mineralized waste is solid waste that has been stored or buried for a certain period of time. While being stored or buried, the solid waste decomposes and generates methane, and the accumulation of methane in the solid waste creates a desirable environment for the natural growth and development of methane-oxidizing organisms therein. In certain embodiments, the mineralized waste is stored in one or more containing devices or facilities for a certain period of time to allow methane-oxidizing organisms to develop. In certain embodiments, the mineralized waste is buried at waste landfill sites.

The storage or burial period of mineralized waste is referred to as the “age” of the mineralized waste. Methane-oxidizing organisms accumulate and grow in mineralized waste as the waste ages. In certain embodiments, the mineralized waste has been aged for a certain period of time so that it no longer releases any leachate or methane gas by itself. In certain embodiments, the mineralized waste has been aged for about 2 to about 30 years, for about 2 to about 25 years, for about 2 to about 20 years, for about 2 to about 15 years, for about 2 to about 10 years, for about 2 to about 8 years, or for about 2 to about 5 years. In certain embodiments, the mineralized waste has been aged for about 5 to about 30 years, for about 5 to about 25 years, for about 5 to about 20 years, for about 5 to about 15 years, for about 5 to about 10 years, for about 5 to about 8 years, or for about 8 to about 10 years. In certain embodiments, the mineralized waste has been aged for at least 1 year, or at least 2 years, or at least 3 years, or at least 4 years, or at least 5 years, or at least 6 years, or at least 7 years, or at least 8 years, or at least 9 years, or at least 10 years, or at least 15 years, or at least 20 years, or at least 25 years, or at least 30 years.

Methane-oxidizing organisms may be artificially introduced to solid waste to form mineralized waste. For example, in certain illustrative embodiments, aged waste from landfills may be taken and dissolved in a nutrient solution as described in Example 1 below. The mixture is incubated for a suitable time period to allow the methane-oxidizing organisms from the waste grow, and the methane-oxidizing organisms are collected from the culture. The collected methane-oxidizing organisms are allowed to grow again in the nutrient solution until a desired concentration is reached. For another example, in certain illustrative embodiments, known strains of methane-oxidizing organisms purchased commercially from an organization such as American Type Culture Collection (ATCC) (for example, Methylococcus capsulatus, ATCC19069) may be cultured in a laboratory until a desired concentration is reached. The methane conversion rate of the methane-oxidizing organism may be measured by placing the methane-oxidizing organisms in a container having an inlet gas port and an outlet gas port. Methane gas is passed into the container through the inlet gas port and gas from the container is collected from the outlet port. The ratio of methane concentration of the inlet gas and that of the outlet gas which has passed through the methane-oxidizing organism is calculated. In certain embodiments, methane-oxidizing organisms may be cultured in laboratories and added into solid waste. In certain embodiments, methane-oxidizing organisms may be isolated from aged solid waste, cultured to a suitable concentration, and added into solid waste. In certain embodiments, solid waste containing methane-oxidizing organisms may be added to fresh waste to make mineralized waste to be used in the system of the present disclosure. In certain embodiments, mineralized waste that has been aged at different places or for different periods of time may be mixed together to make the mineralized waste to be used in the system of the present disclosure.

The mineralized waste may be measured for its capacity to oxidize methane into other substances by measuring and calculating its methane conversion rate. The methane conversion rate over a period of time t (t=t₁−t₀) may be calculated using the following equation:

$$Methane Conversion Rate = 1 - \frac{Methane Concentation at {t}_{1}}{Inlet Methane Concentration at {t}_{0}}$$

The methane conversion rate may be determined by measuring the methane concentrations at time t₀ and t₁ and then calculating the rate using the equation above. An illustrative embodiment of a method for measuring and calculating the methane conversion rate is described in Example 1 below.

In certain embodiments, the mineralized waste has a methane conversion rate over a period of 24 hours of at least 50%, or at least 60%, or at least 70%, or at least 80%, or at least 85%, or at least 90%, or at least 95%, or at least 97%, or at least 98%, or at least 99%. In certain embodiments, the mineralized waste has a methane conversion rate over a period of 24 hours of between 50% and 100%, between 50% and 90%, between 50% and 80%, between 50% and 70%, between 50% and 60%, between 60% and 100%, between 70% and 100%, between 80% and 100%, and between 90% and 100%. In certain embodiments, the mineralized waste has a methane conversion rate over a period of 48 hours of at least 50%, or at least 60%, or at least 70%, or at least 80%, or at least 85%, or at least 90%, or at least 95%, or at least 97%, or at least 98%, or at least 99%. In certain embodiments, the mineralized waste has a methane conversion rate over a period of 48 hours of between 50% and 100%, between 50% and 90%, between 50% and 80%, between 50% and 70%, between 50% and 60%, between 60% and 100%, between 70% and 100%, between 80% and 100%, and between 90% and 100%.

Water content of the mineralized waste may affect the oxidation efficiency of methane. In certain embodiments, the mineralized waste has water content between 5% and 30%, between 5% and 25%, between 5% and 20%, between 5% and 15%, between 5% and 10%, or between 5% and 8%. In certain embodiments, the mineralized waste has water content between 10% and 30%, between 10% and 25%, between 10% and 20%, or between 10% and 15%. In certain embodiments, the mineralized waste has water content between 15% and 30%, between 15% and 25%, or between 15% and 20%. The water content of the mineralized waste can be adjusted to appropriate levels by any suitable method. In certain embodiments, mineralized waste may be dried to reduce the water content. Drying methods may include, without limitation, natural drying methods such as by air or sun, or drying by machines, or any combination thereof, or any other suitable method. In certain embodiments, mineralized waste may be wetted to increase the water content. Such wetting methods may include, without limitation, water spraying, wetting, and humidification. In certain embodiments, the mineralized waste is wetted to suitable water content before added into the system. In certain embodiments, water is sprayed onto the mineralized waste in the process of filling the mineralized waste into the system.

In certain embodiments, the mineralized waste is sifted by suitable sieves to remove rocks, stones, glass, rubber, plastic, wood sticks, and other large debris contained therein and to help break up the large lumps of mineralized waste into smaller parts. In certain embodiments, the mineralized waste is sifted by a suitable sieve to obtain mineralized waste having particles and soils of a suitable size. The mesh sizes of the sieves can be selected so that the undesirable components in the mineralized waste can be separated from the desirable components. In certain embodiments, mineralized waste is sifted by 0.3-0.5 cm sieves, 0.5-1 cm sieves, 1-2 cm sieves, 2-4 cm sieves, 4-6 cm sieves, or 6-8 cm sieves. In certain embodiments, the mineralized waste is smashed into small pieces or ground into fine particles before being sifted.

In certain embodiments, the mineralized waste may further include a nutrient solution selected to promote the growth of methane-oxidizing organisms. The nutrient solution may contain various ingredients dissolved in water or buffer that can promote growth and activity of methane-oxidizing organisms. In certain embodiments, the nutrient solution may contain phosphate, nitrate, or sulfate, or any combination thereof. pH value of the nutrient solution may be in a suitable range for growth of the methane-oxidizing organisms. In an illustrative embodiment, the pH value of the nutrient solution is between 5 and 9, between 6 and 8, between 6.1 and 7.9, between 6.2 and 7.8, between 6.3 and 7.7, between 6.4 and 7.6, between 6.5 and 7.5, between 6.6 and 7.4, between 6.7 and 7.3, between 6.8 and 7.2, and between 6.9 and 7.1. In another illustrative embodiment, the pH value of the nutrient solution is 7. In an illustrative embodiment, the nutrient solution includes phosphate and nitrate. In an illustrative embodiment, the nutrient solution includes phosphate and sulfate. In an illustrative embodiment, the nutrient solution includes nitrate and sulfate. In an illustrative embodiment, the nutrient solution includes 1-20 g/l phosphates, 0.3-9 g/l nitrates, and/or 0.05-3 g/l sulfates in concentration, and then may be applied to the waste at a volume (ml) to weight (g) ratio of the nutrient solution to the mineralized waste between 1 ml per 100 g and 50 ml per 100g, between 3 ml per 100 g and 50 ml per 100 g, between 5 ml per 100 g and 50 ml per 100 g, between 10 ml per 100g and 50 ml per 100 g, between 20 ml per 100 g and 50 ml per 100 g, between 30 ml per 100 g and 50 ml per 100 g, between 40 ml per 100 g and 50 ml per 100 g, between 1 ml per 100 g and 40 ml per 100 g, between 1 ml per 100 g and 30 ml per 100 g, 1 ml per 100 g and 25 ml per 100 g, between 1 ml per 100 g and 20 ml per 100 g, and between 1 ml per 100 g and 10 ml per 100 g. In another illustrative embodiment, the weight ratio of the nutrient solution to the waste is 4 ml per 100 g to 6 ml per 100 g. In an illustrative embodiment, the nutrient solution includes 1-10 g/l phosphates, 0.3-9 g/l nitrates, and/or 0.05-3 g/l sulfates in final concentration when applied to the waste. In an illustrative embodiment, the nutrient solution includes 1-7 g/l phosphates, 0.3-9 g/l nitrates, and/or 0.05-3 g/l sulfates in final concentration when applied to the waste. In an illustrative embodiment, the nutrient solution includes monopotassium phosphate, disodium phosphate, dipotassium phosphate, monosodium phosphate, calcium phosphate, magnesium phosphate, ferrous phosphate, ferric phosphate, sodium nitrate, potassium nitrate, calcium nitrate, magnesium nitrate, ferrous nitrate, ferric nitrate, potassium sulfate, sodium sulfate, magnesium sulfate, calcium sulfate, ferrous sulfate, and/or ferric sulfate. In an illustrative embodiment, the nutrient solution includes monopotassium phosphate and/or disodium phosphate. In an illustrative embodiment, the nutrient solution includes sodium nitrate. In an illustrative embodiment, the nutrient solution includes potassium sulfate, magnesium sulfate and/or ferrous sulfate. In an illustrative embodiment, the nutrient solution further includes some trace elements selected from the group consisting of ZnSO₄, MnSO₄, H₃BO₃, Na₃MoO₄, CoCl₂, KI, and CaCl₂. The concentration of these trace elements in the nutrient solution can be less than 100 ug/l, less than 80 ug/l, less than 70 ug/l, less than 60 ug/l, less than 50 ug/l, less than 40 ug/l, less than 30 ug/L, less than 20 ug/l, and less than 10 ug/l. The concentration of these trace elements in the nutrient solution can be between 10 ug/l and 100 ug/l, 10 ug/l and 50 ug/l, 50 ug/l and 100 ug/l, and 1 ug/l and 10 ug/l.

In certain embodiments, the mineralized waste may further include mineralized sludge. The term “mineralized sludge” refers to waste sludge that contains methane-oxidizing organisms such as bacteria and archaea. The methane-oxidizing organisms can be introduced to the mineralized sludge through natural growth and development or in an artificial manner. For example, methane-oxidizing organisms may be purchased commercially or cultured in a laboratory using strains isolated from aged waste as described before. The methane conversion rate of the methane-oxidizing organism may be measured by placing the methane-oxidizing organisms in a container having an inlet gas port and an outlet gas port. Methane gas is passed into the container through the inlet gas port and gas from the container is collected from the outlet port. The ratio of methane concentration of the inlet gas and that of the outlet gas which has passed through the methane-oxidizing organism is calculated. In certain embodiments, methane-oxidizing organisms may be cultured in laboratories and added into waste sludge. In certain embodiments, methane-oxidizing organisms may be isolated from aged solid waste, cultured to a suitable concentration, and added into waste sludge. In certain embodiments, solid waste containing methane-oxidizing organisms may be added to waste sludge to make mineralized sludge to be used in the system of the present disclosure. In certain embodiments, mineralized sludge that has been aged at different places or for different periods of time may be mixed together to make the mineralized sludge to be used in the system of the present disclosure.

In certain embodiments, the mineralized sludge is aged for a certain period of time to allow methane-oxidizing organisms to grow and develop. The mineralized sludge may be buried under the ground or stored in one or more storage devices or facilities to be aged. In certain embodiments, the mineralized sludge is aged for 2 to 30 years, for 2 to 25 years, for 2 to 20 years, for 2 to 15 years, for 2 to 10 years, for 2 to 8 years, or for 2 to 5 years. In certain embodiments, the mineralized sludge is aged for 5 to 30 years, for 5 to 25 years, for 5 to 20 years, for 5 to 15 years, for 5 to 10 years, or for 5 to 8 years. In certain embodiments, the mineralized sludge is aged for at least 1 year, or at least 2 years, or at least 3 years, or at least 4 years, or at least 5 years, or at least 6 years, or at least 7 years, or at least 8 years, or at least 9 years, or at least 10 years, or at least 15 years, or at least 20 years, or at least 25 years, or at least 30 years.

The mineralized sludge can have suitable water content. In certain embodiments, the mineralized sludge has a water content ranging from 30% to 60%, from 30% to 55%, from 30% to 50%, from 30% to 45%, from 30% to 40%, or from 30% to 35%. In certain embodiments, the mineralized sludge has a water content ranging from 40% to 60%, from 40% to 55%, from 40% to 50%, or from 40% to 45%.

The mineralized waste may contain the mineralized sludge in any suitable mixing ratio. In certain embodiments, the weight percentage of mineralized sludge in the mineralized waste is from about 0% to about 50%, or from about 0% to about 40%, or from about 0% to about 30%, or from about 0% to about 20%, or from about 0% to about 10%, or from about 40% to about 50%, or from about 30% to about 50%, from about 20% to about 50%, or from about 10% to about 50%. The suitable percentage of mineralized sludge contained in the mineralized waste may be determined by a person skilled in the art.

In certain embodiments, the layer of mineralized waste has a thickness of about 0.2 m to about 2 m, or about 0.2 m to 1.5 m, or about 0.2 m to 1 m, or about 0.2 m to 0.8 m, or about 0.4 m to about 2 m, or about 0.4 m to about 1.5 m, or about 0.4 m to about 1 m, and about 0.4 to about 0.8 m. In certain embodiments, the layer of mineralized waste has a thickness of at least 0.2 m, or at least 0.3 m, or at least 0.4 m, or at least 0.5 m, or at least 0.6 m, or at least 0.7 m, or at least 0.8 m, or at least 0.9 m, or at least 1 m, or at least 1.5 m.

In certain embodiments, the layer of mineralized waste surrounding the at least one conduit has a thickness proportional to the size of the at least one conduit. In certain embodiments, the ratio of the thickness of the mineralized waste to the diameter of the at least one conduit is between about 0.5 to about 6, between about 0.5 to about 5, between about 0.5 to about 4, between about 0.5 to about 3.5, between about 0.5 to about 3, between about 0.5 to about 2.5, between about 0.5 to about 2, between about 0.5 to about 1.5, or between about 0.5 to about 1. In certain embodiments, the ratio of the thickness of the mineralized waste to the diameter of the at least one conduit is between about 1 to about 10, between about 1 to about 8, between about 1 to about 6, between about 1 to about 5, between about 1 to about 4, between about 1 to about 3, between about 1 to about 2, or between about 1 to about 1.5.

The system for oxidizing methane may include a construct with at least one conduit configured for air access. The construct may have more than one conduit. When the construct has more than one conduit, the conduits may or may not be connected to each other. The one or more conduits can be configured for air access by any suitable means. For example, in certain illustrative embodiment, the construct consists of one or more tubes that extend out from the waste into the space outside of the waste and thus allow gas release into the environment. For another example, in certain illustrative embodiment, the construct consists of one or more tubes wherein each tube contains an air filter at its end which can absorb methane. For another example, in certain illustrative embodiment, the construct consists of one or more gabions buried in the waste with at least one end sticking out of the waste. In certain embodiments, one or more conduits of the construct may extend out of the waste to allow gas release. In certain embodiments, one or more conduits of the construct may be connected to a device in such a way that may further filter or process the gas from the waste before it is released into the atmosphere.

The conduits may have any suitable shape that would allow air flow within or through the conduits. The conduits may be pipes, tubes, ducts, channels or other suitable structures, and may be straight, bended, or branched. The cross-section of any conduit may be circular, triangular, square, rectangular, or any other regular or irregular shape. In certain embodiments, the conduits are cylindrical pipes or tubes. In certain embodiments, the cylindrical pipes and tubes have diameters between 10 cm and 100 cm, between 20 cm and 100 cm, between 30 cm and 100 cm, between 40 cm and 100 cm, between 50 cm and 100 cm, between 5 cm and 50 cm, between 10 cm and 50 cm, between 20 cm and 50 cm, between 30 cm and 50 cm, or between 40 cm and 50 cm.

The one or more conduits may be made of any suitable material. Illustrative examples of suitable materials for the conduits are metal such as steel, iron, copper and alloy, plastic, rubber, glass, cement, and stone. In certain embodiments, the one or more conduits are made of air-permeable materials. In certain embodiments, the one or more conduits may be perforated. In certain embodiments, one or more conduits are perforated along the whole length of the conduits. In certain embodiments, one or more conduits of the construct are perforated for two thirds of its length, one half of its length, or one third of its length, or one fourth of its length. In certain embodiments, one or more conduits are perforated for the lower two thirds of its length, or for the lower one half of its length, or for the lower one third of its length, or for the lower one fourth of its length. In certain embodiments, one or more conduits are perforated for the higher two thirds of its length, or for the higher one half of its length, or for the higher one third of its length, or for the higher one fourth of its length.

In certain embodiments, each conduit of the construct is surrounded by a layer of mineralized waste. In certain embodiments, one or more conduits of the construct are surrounded by a layer of mineralized waste. In certain embodiments, one or more conduits of the construct are surrounded by more than one layer of mineralized waste.

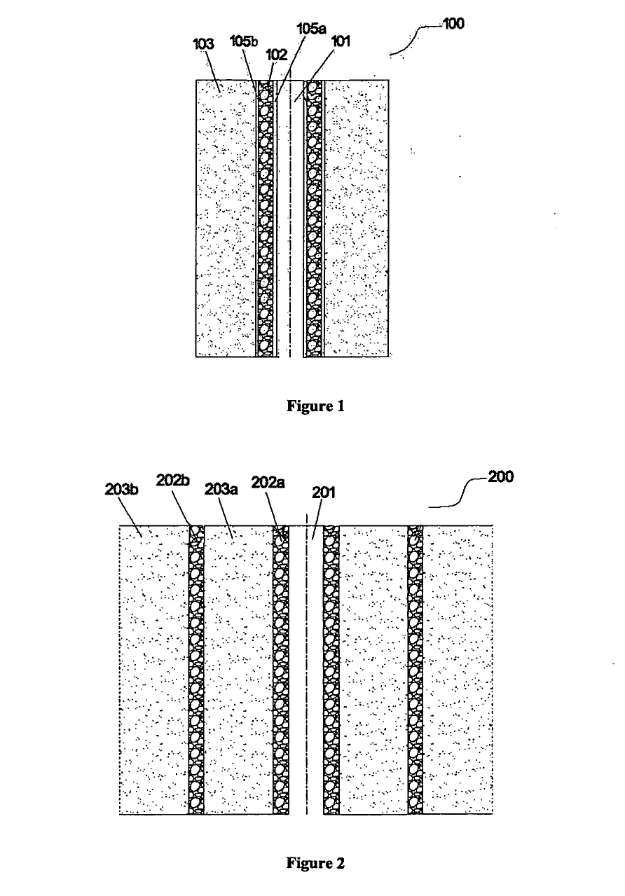

In certain embodiments, the system may include a separation layer between a conduit and the layer of mineralized waste surrounding it. The separation layer can be configured to facilitate air circulation and air flow between the conduit and the layer of mineralized waste. In certain embodiments, the separation layer has a thickness of about 100 mm to about 500 mm, about 100 mm to about 400 mm, about 100 mm to about 300 mm, and about 100 mm to about 200 mm. In certain embodiments, the separation layer has a thickness of at least 50 mm, or at least 60 mm, or at least 70 mm, or at least 80 mm, or at least 90 mm, or at least 100 mm, or at least 120 mm, or at least 150 mm. In an illustrative embodiment shown in FIG. 1, the system 100 for oxidizing methane contains a conduit 101 for air access, a separation layer 102, and a layer of mineralized waste 103.

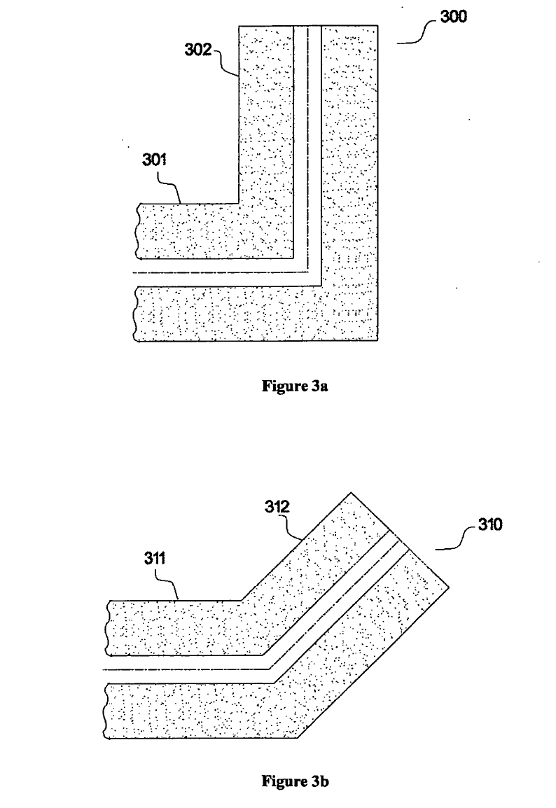

In certain embodiments, the system may include a plurality of separation layers and a plurality of layers of mineralized waste surrounding one or more conduits, in which each layer of mineralized waste is adjacent to at least one separation layer. In certain embodiments, the system may include a plurality of separation layers and a plurality of layers of mineralized waste, in which each separation layer is positioned between a conduit and a layer of mineralized waste or between two adjacent layers of mineralized waste or between a layer of mineralized waste and the materials to be oxidized. In an illustrative embodiment shown in FIG. 2, the system 200 for oxidizing methane contains a conduit 201, a first separation layer 202a between the conduit 201 and a first layer of mineralized waste 203a, and a second separation layer 202b outside of the first layer of mineralized waste 203a, and a second layer of mineralized waste 203b outside of the second separation layer 202b.

The one or more separation layers may contain any suitable material configured for facilitating air circulation. Illustrative examples of the materials in a separation layer are stone particles, wood sticks, plastic debris, gravels, tire shreds, broken glasses, and any combination thereof. In certain embodiments, the separation layer contains gravels, tire shreds, and/or broken glasses.

In certain embodiments, the separation layer is composed of gravels. In certain embodiments, the gravels have a diameter of about 50 mm to about 150 mm, about 50 mm to about 140 mm, about 50 mm to about 120 mm, about 50 mm to about 100 mm, or about 50 mm to about 80 mm. In certain embodiments, the gravels have a diameter of no less than 40 mm, 50 mm, 60 mm, 70 mm or 80 mm, and no more than 100 mm, 110 mm, 120 mm, 130 mm, 140 mm or 150 mm.

In certain embodiments, the gravels in the separation layer may be in random distribution. In certain embodiments, the gravels in the separation layer may be arranged in such a way that promotes air exchange. In certain embodiments, the gravels may be arranged with the larger sized gravels close to the center of the conduit that is surrounded by the separation layer. In certain embodiments, the layer of gravels have a thickness of about 100 mm to about 500 mm, about 100 mm to about 400 mm, about 100 mm to about 300 mm, and about 100 mm to about 200 mm. In certain embodiments, the layer of gravels have a thickness of at least 50 mm, or at least 60 mm, or at least 70 mm, or at least 80 mm, or at least 90 mm, or at least 100 mm, or at least 120 mm, or at least 150 mm.

In certain embodiments, the system further includes one or more dividers for separating the different structures of the system. In certain embodiments, the system includes a divider between a conduit and a layer of mineralized waste surrounding it. In certain embodiments, the system includes a divider between a conduit and a separation layer surrounding it. In certain embodiments, the system includes a divider between each layer of mineralized waste and each conduit surrounded by it. In certain embodiments, the system includes a divider between each separation layer and each conduit surrounded by it. In certain embodiments, the system includes a divider between a separation layer and a layer of mineralized waste adjacent to the separation layer. In certain embodiments, the system includes a divider between each layer of mineralized waste and each separation layer. In certain embodiments, the system includes a divider surrounding a layer of mineralized waste. In certain embodiments, the system includes a divider surrounding a separation layer. In an illustrative embodiment shown in FIG. 1, the system 100 contains a divider 105a between the conduit 101 and the separation layer 102, and a divider 105b between the separation layer 102 and the layer of mineralized waste 103.

The divider can be configured to be air-permeable. Any suitable material may be used to make the divider. Illustrative embodiments of the suitable materials are metal, plastic, rubber, stone, glass, fabric, and so on. In certain embodiments, the divider is porous or perforated. In certain embodiments, the divider is a steel net, mesh, sand net, wire netting, perforated metal, porous plastic, or gabion.

The system may be used at any place where there is a need for methane oxidation. In certain embodiments, the system is used for oxidizing methane at a solid waste landfill site. In certain embodiments, the system is used at a storage place for waste. In certain embodiments, the system is used at an area or a place where there is a high concentration of methane. In certain embodiments, the construct for air access is installed in the waste site before the layer of mineralized waste and/or the separation layer is installed. In certain embodiments, the construct for air access is installed in the waste site after the layer of mineralized waste and/or the separation layer is installed. In certain embodiments, the construct for air access and the layer of mineralized waste and/or the separation layer are installed together into the waste site.

In another aspect, the present disclosure provides a method for oxidizing methane, which includes putting a system for oxidizing methane into contact with the material to be oxidized, in which the system includes a construct with at least one conduit configured for air access, and a layer of mineralized waste surrounding the at least one conduit.

The material to be oxidized can be any material that contains or generates methane. The material to be oxidized may include, without limitation, solid waste and sewage waste. In certain embodiments, the material to be oxidized is solid waste.

The system may be placed in contact with the material to be oxidized in any suitable manner. In certain embodiments, the system for oxidizing methane is placed substantially vertically in the material to be oxidized. In certain embodiments, the system for oxidizing methane is placed slantingly in the material to be oxidized. In certain embodiments, the system is placed substantially horizontally in the material to be oxidized. The term “substantially vertically” means that the longitudinal axis at about the center of the construct for air access is at an angle of about 90° to the ground. The term “slantingly” means that the longitudinal axis at about the center of the construct for air access is at an angle between 0° and 90° to the ground. The term “substantially horizontally” means that the longitudinal axis at about the center of the construct for air access is at angle of about 0° to the ground.

The system may have different portions wherein each portion is oriented at a different angle relative to the ground. In certain embodiments, the system has a portion oriented substantially vertically, a portion oriented slantingly, and/or a portion oriented substantially horizontally. In an illustrative embodiment shown in FIG. 3a, the system 300 has a first portion 301 that is oriented substantially horizontally to the ground, and a second portion 302 that is oriented substantially vertically to the ground. In another illustrative embodiment shown in FIG. 3b, the system 310 has a first portion 311 that is oriented substantially horizontally to the ground, and a second portion 312 that is oriented slantingly to the ground. In another illustrative embodiment shown in FIG. 3c, the system 320 has a first portion 321 oriented substantially horizontally, a second portion 322 oriented slantingly, and a third portion 323 oriented substantially vertically. In another illustrative embodiment shown in FIG. 3d, the system 330 has a U shape. The different portions may be made as a continuous integral structure or may be assembled from separate parts. Each portion may have the same internal structure, for example, each portion may contain one air conducting pipe surrounded by one layer of mineralized waste and one separation layer. Alternatively, different portions may have different internal structure, for example, one portion may have one air conducting pipe surrounded by one layer of mineralized waste and one separation layer, and another portion may have two layers of mineralized waste separated by a separation layer, and an air conducting pipe surrounded within the first layer of mineralized waste.

In certain embodiments, the construct with at least one conduit for air access has at least one end extended out of the material to be oxidized. In certain embodiments, the system for oxidizing methane is placed substantially vertically or slantingly into the material to be oxidized, and the top of the at least one conduit for air access is higher than the top surface of the material to be oxidized. In certain embodiments, the system is placed horizontally in the material to be oxidized, and at least one end of the at least one conduit for air access is extended out of the side of the material to be oxidized.

In certain embodiments, the material to be oxidized may partially or completely surround the portion of the system that contains mineralized waste. In certain embodiments, the material to be oxidized may at least partially surround the system. In certain embodiments, the method further includes adding additional mineralized waste to the layer of mineralized waste. In certain embodiments, the method includes adding additional mineralized waste to the layer of mineralized waste when material to be oxidized is added. In certain embodiments, the method includes adding additional mineralized waste into the layer of mineralized waste in order to keep the height of the mineralized waste at substantially the same level as the material to be oxidized when the system for oxidizing methane is placed substantially vertically or slantingly into the material to be oxidized.

In certain embodiments, the method includes adding a nutrient solution into the layer of mineralized waste. In certain embodiments, the method includes spraying the nutrient solution onto the layer of mineralized waste for every 1-2 meters of mineralized waste in height added to the layer of mineralized waste. In certain embodiments, the method includes spraying the nutrient solution onto the surface of the layer of mineralized waste when the layer of mineralized waste reaches a desirable height. In certain embodiments, the method includes mixing the nutrient solution with the mineralized waste before filing the mineralized waste to the system. The amount of nutrient solution added to the layer of mineralized waste can be selected as appropriate by a person skilled in the art. In certain embodiments, for every 100 g of the mineralized waste, about 1-100 ml nutrient solution is added, wherein, every 1 liter of nutrient solution contains 1-20 g phosphates, 0.3-9 g nitrates, 0.05-3 g sulfates, less than 100 ug ZnSO₄, less than 100 ug MnSO₄, less than 100 ug H₃BO₃, less than 100 ug Na₃MoO₄, less than 100 ug CoCl₂, less than 100 ug KI, and less than 100 ug CaCl₂.

### EXAMPLES

The following Examples are set forth to aid in the understanding of the present disclosure, and should not be construed to limit in any way the scope of the invention as defined in the claims which follow thereafter.

### Example 1

The solid waste from the Laogang landfill in Shanghai is used for making mineralized waste for setting up a system for oxidizing methane. The mineralized waste is made by mixing solid waste deposited at the landfill in 1992 and solid waste deposited at the landfill in 2001 at the ratio of 7:3 (w/w). The mineralized waste is smashed into smaller pieces and sifted using large size sieves. The materials that sift through the sieves are collected. The collected waste is further broken into fine materials and sifted using 2-4 cm sieves. The materials that sift through the sieves are collected as the mineralized waste to be used in the system. Table 1 shows the chemical properties of the mineralized waste used in the experiment.

TABLE 1
Parameters of the mineralized waste

| | Chemical Property | | | | | | | |
|---|---|---|---|---|---|---|---|---|
| | | | | Cation exchange | | Total | Total | Total |
| | | Water | Organic | capacity | | Nitrogen | Phosphor | Potassium |
| | | content | substance | (mg/100 g | Conductivity | Content | Content | Content |
| | pH | (%) | content(g/kg) | dry waste) | (μS/cm) | (g/kg) | (g/kg) | (g/kg) |
| Mineralized | 7.80 | 15.50 | 102.5 | 65.4 | 763.3 | 4.68 | 7.25 | 8.68 |
| waste | | | | | | | | |

A nutrient solution, i.e. the NMS solution, is added to the mineralized waste. The formulation of 1L NMS solution includes: 1.06 g KH₂PO₄, 4.34 g Na₂HPO₄.12H₂O, 1.70 g NaNO₃, 0.34 g K₂SO₄, 0.074 g MgSO₄.7H₂O, 22.4 mg FeSO₄.7H₂O, and 2 mL trace elements solution. The pH of the solution is 7.0. The composition of the trace elements solution is shown in Table 2 below. 6 g NMS solution is added to every 100 g of mineralized waste.

TABLE 2
The composition of the trace elements solution

| | Components | | | | | | |
|---|---|---|---|---|---|---|---|
| | ZnSO₄•7H₂O | MnSO₄•7H₂O | H₃BO₃ | Na₂MoO₄•2H₂O | CoCl₂•6H₂O | KI | CaCl₂•2H₂O |
| Concentration | 0.57 | 0.446 | 0.124 | 0.096 | 0.096 | 0.166 | 7.0 |
| (mg/L) | | | | | | | |

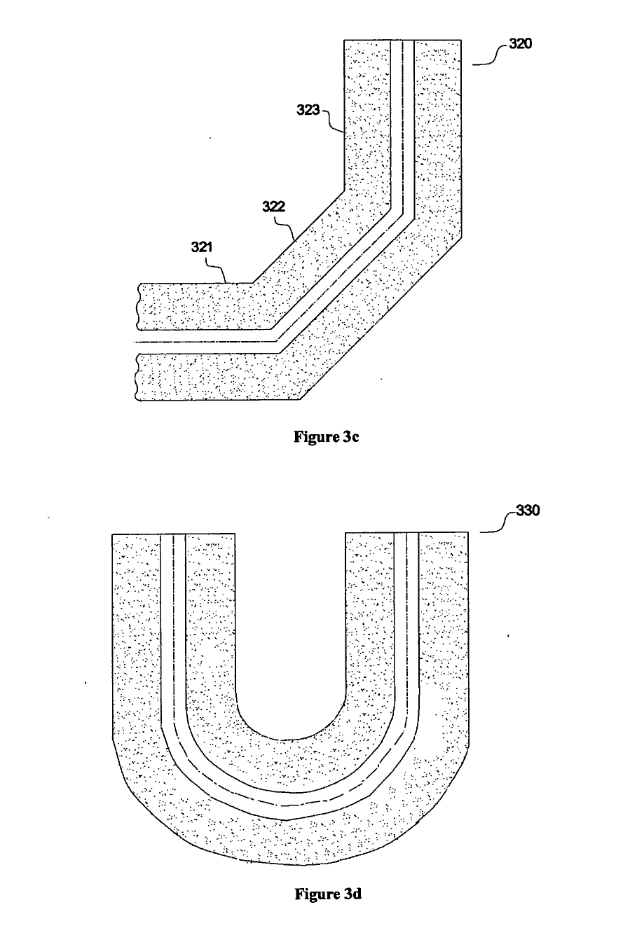

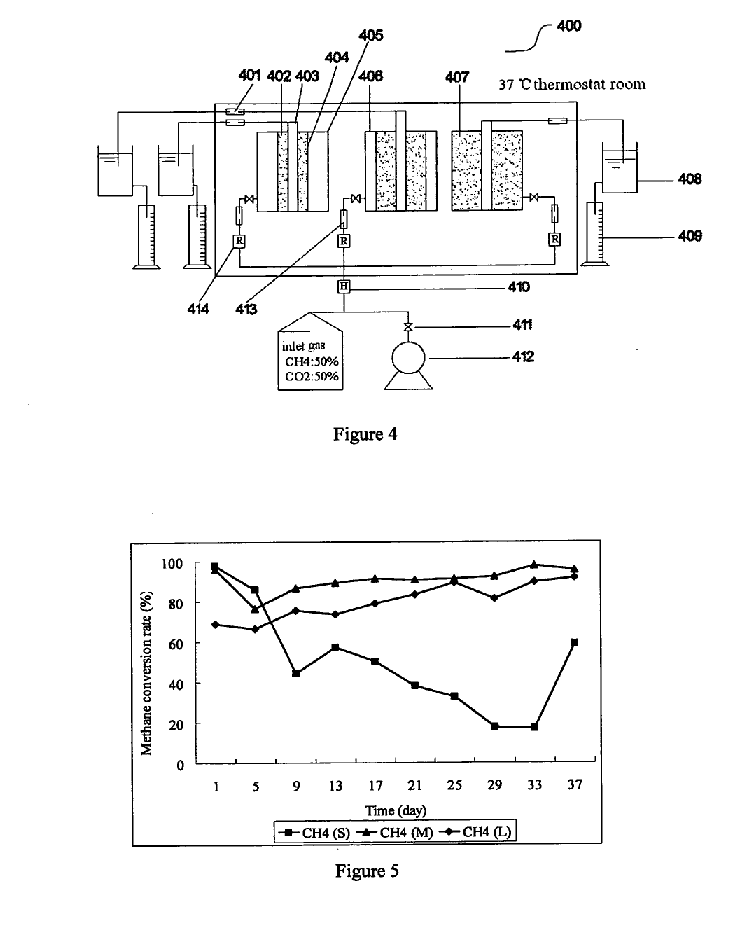

A system for oxidizing methane—system 400—is set up as shown in FIG. 4. Three plastic barrels (with lids) are used in the experiment, in which 405 represents a small sized barrel having the diameter of 80 mm; 406 represents a medium sized barrel of 160 mm, and 407 represents a large sized barrel of 240 mm. The barrels are 35 cm in height, 26 cm in diameter, and 18 L in volume. An airway pipe 403 with a diameter of 40 mm and a height of 0.4 m is placed vertically at the center of each barrel. The top of the airway pipe extends out of the top of each barrel. The airway pipes are perforated at the lower ⅔ portion to facilitate gas flow into the pipe. The upper ⅓ portion of the airway pipe is not perforated so that gas may accumulate there. Each airway pipe is wrapped with sand net to prevent jamming of the perforations in the airway pipe by the particles of mineralized waste. Gabions 404 with a diameter of 80 mm, 160 mm, and 240 mm are placed surrounding the three airway pipes, respectively. The diameters of the gabions are 2, 4 and 6 times, respectively, of the diameters of the airway pipes.

The layer of mineralized waste 402 is filled into the space between the wrapped airway pipe and the gabion in each barrel. The thicknesses of the mineralized waste in the three gabions are 20 mm, 60 mm, and 100 mm, respectively. The thickness of the mineralized waste is the difference between the diameter of the mineralized waste column and the diameter of the airway pipe. The diameter of the mineralized waste column is the horizontal distance from the center of the airway pipe to the outer boundary of the gabion. For every 100 g mineralized waste filled into the gabion, 6 g nutrient solution is sprayed onto the surface of the mineralized waste.

A gas inlet valve is set at the lower part of the outer wall of each barrel for the intake of methane and air. Methane is obtained from the gas mixture (CH₄:CO₂=1:1) in a steel cylinder. Air is pumped in by an aerobic pump 412 controlled by a valve 411. The flow rate of methane at each barrel is measured by a flow meter 414 and is controlled at 6 ml/min and air 60 ml/min. Methane and air are allowed to mix and be humidified in a bottle 410, and the resulting moist mixture of methane and air flows along an inlet pipe and to the inlet valve of the barrels.

Upon opening of the inlet valve, the gas mixture of methane and air enters into the mineralized waste column and diffuses in the mineralized waste. The methane-oxidizing bacteria oxidize methane into carbon dioxide. The remaining gas from the mineralized waste exits from the airway pipe, and is collected in a gas-collecting bottle 408 and measured at a graduated cylinder 409.

The samples of inlet gas and outlet gas are taken at the respective gas pipes using a syringe needle. The samples are measured using a GC-14B gas chromatography equipment manufactured by SHIMADZU.

The experiment lasts for 37 days, and inlet and outlet gas samples are taken at regular time points at the sampling port 413 in the inlet pipe and at the sampling port 401 in the outlet pipe, repectively. Methane concentrations in the inlet gas samples and outlet gas samples are measured. Methane conversion rate is calculated using the following equation:

$$Methane Conversion Rate = 1 - \frac{Outlet Methane Concentation}{Inlet Methane Concentration}$$

FIG. 5 shows the methane conversion rates in the three barrels with mineralized waste columns having different thickness. S (small), M (medium), and L (large) in FIG. 5 represent the mineralized waste columns having the diameter of 80 mm, 160 mm, and 240 mm, respectively. The medium and large sized mineralized waste columns show higher methane conversion rates and more stable oxidation effects than the small sized column during the 37-day study period. The medium sized mineralized waste column shows exceptionally good results, and its methane conversion rate steadily increases with little fluctuation during the study period and finally reaches a plateau of about 95-97%.

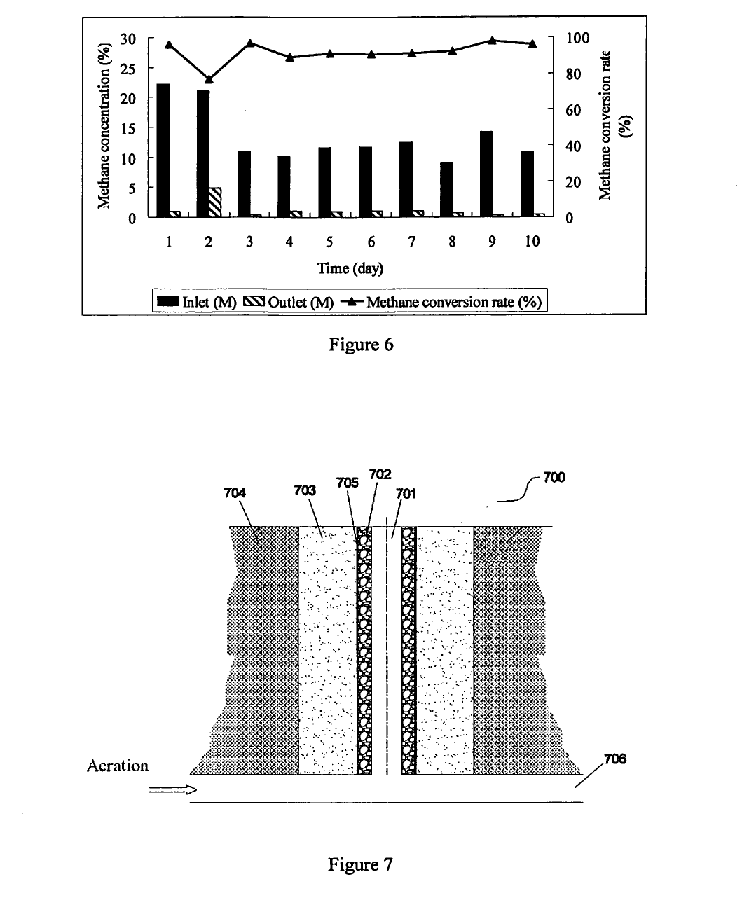

The medium sized mineralized waste column is further tested under different inlet methane concentrations. The experiment lasts for 10 days using the same method as described above, except that different methane concentrations in the inlet gas are used in the experiment. Methane concentrations in inlet gas and outlet gas are measured respectively, and the methane conversion rate is calculated. The results are shown in FIG. 6. The methane conversion rate remains stable under different inlet methane concentrations. Except for slight fluctuation in the beginning of the test, the methane conversion rates during the 10-day study period are almost all above 90%, with the highest rate at 97.8%.

### Example 2

The #42 landfill unit in the Shanghai Laogang landfill site is used for the study. The landfill unit has a total capacity of 10,000 m³, and is divided into two separate units, each unit having an average capacity for 5000 m³. The landfill height is 7 m, and the gradient is 1:1. Each of the two separate units is trapezoidal, and the length and width of the top of each unit is 33.5 m by 33.5 m; the bottom is 9.5 m by 19.5 m. The two units are separated by a garbage dam, and the width of the top of the dam is 1 m. The bottom and side of the unit is treated with two layers of 200 g/m² geotextile fabric and one layer of 1.5 mm HDPE membrane to avoid leaks. Leachate pipes and airway pipes for derivative gas are set up in the units.

One of the two units is used as the control unit, and the other unit is used as the experimental unit for methane emission reduction study.

The control unit is prepared as summarized here. The unit is filled with 5,000 m³ fresh garbage, and then covered with mud of 0.6 m in thickness (500 m³). The surface is covered with a plastic sheet. A leachate collection pipe (diameter 250 mm, length 55 m) is placed at the bottom of the unit. An airway pipe (De250, 7.8 m) for derivative gas is placed at the mid-point of the leachate collection pipes (the airway pipe is not connected with the leachate collection pipe). The landfill leachate collection pipes extend out of the landfill units and connect with the leachate collection barrels.

The experimental unit 700 is prepared as summarized here. The landfill is prepared in the same way as in the control unit, except that the leachate collection pipe 706 is connected with the airway pipe 701, and that a gabion 702 of 1.25 m in diameter is placed surrounding the airway pipe to fence out the fresh waste 704 in the surrounding area. Inside the gabion, gravels having a size of 50-150 mm are filled surrounding the airway pipe, forming an inner layer of 100 mm in thickness inside the gabion; mineralized waste 703 is filled in the space between the gravel layer and the gabion, forming an outer layer of about 400 mm in thickness inside the gabion. The mineralized waste is sieved and particles having a particle diameter below 4 cm are used in the study. The gravel layer and the mineralized waste layer are separated by a sand net 705. A schematic cross-sectional drawing of the system is shown in FIG. 7. When fresh waste is added into the landfill, mineralized waste is added into the gabion so that the height of the mineralized waste in the gabion is kept the same as that of the fresh waste in the surrounding area.

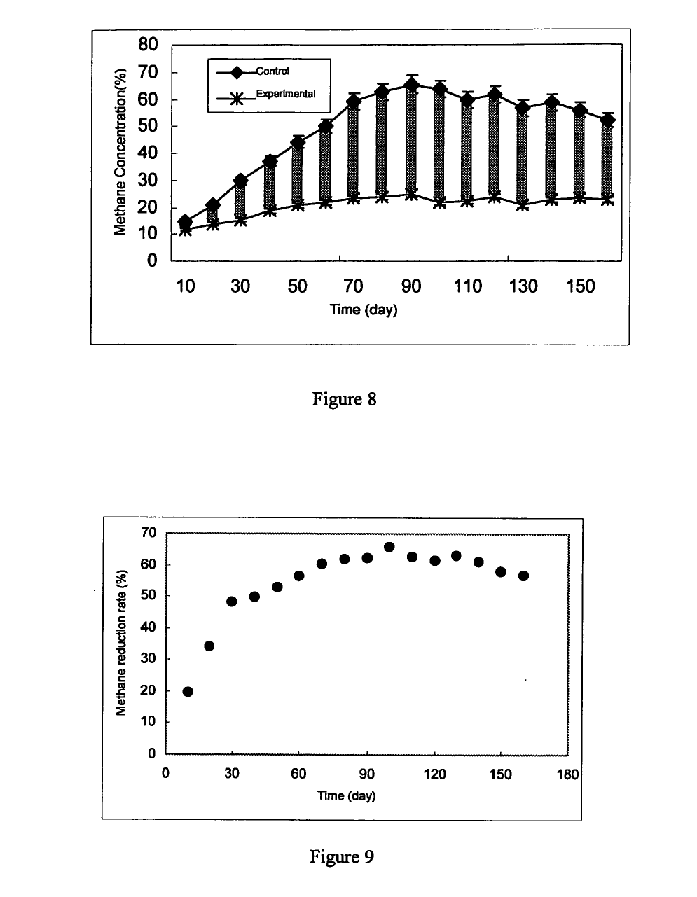

The experiment is conducted for more than 150 days. Gas exiting from the airway pipes of the two units is sampled and analyzed by a portable gas analyzer at various time points during the experiment period. The results are shown in FIG. 8 and FIG. 9.

FIG. 8 shows that the methane concentration in the outlet gas is significantly reduced in the experimental unit than in the control unit. During the experiment period, the methane concentration in the control unit reaches the highest concentration of above 60%, and stays around about 60% during the last three months. However, for the experimental unit having mineralized waste, the methane concentration is below 25% throughout the study, with much smaller fluctuation, indicating that the system provided herein is less affected by the surrounding environment.

FIG. 9 demonstrates the reduction of methane emission by the experimental unit as compared with the control unit. The control unit represents the natural emission of methane in a landfill. Methane reduction rate in the experimental unit is calculated with respect to the methane concentration in the control unit by the following equation:

$$Methane reduction rate = \frac{\begin{matrix}Control Unit Methane Conc . - \\ Experimental Unit Methane Conc\end{matrix}}{Control Unit Methane Conc .}$$

FIG. 9 shows that the methane reduction rate stays at around 60% after a rapid growth, and reaches the maximum reduction rate of about 65% after about 3 months. The results show that the experimental unit significantly reduces the emission of methane in the landfill.

General

With respect to the use of substantially any plural and/or singular terms herein, those having skill in the art can translate from the plural to the singular and/or from the singular to the plural as is appropriate to the context and/or application.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells and so forth.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system for oxidizing methane comprising:
a construct with at least one conduit configured for air access; and
a layer of mineralized waste configured to surround the at least one conduit.

2. The system of claim 1, wherein the mineralized waste has been aged for 5 to 30 years.

3. The system of claim 1, wherein the mineralized waste has water content between 10% and 30%.

4. The system of claim 1, wherein the mineralized waste has been sifted by 2-4cm sieves.

5. The system of claim 1, wherein the mineralized waste further includes a nutrient solution.

6. (canceled)

7. The system of claim 1, wherein the mineralized waste further includes mineralized sludge.

8. The system of claim 1, wherein the thickness of the layer of mineralized waste is about 0.4 m to about 2 m.

9. The system of claim 1, wherein the at least one conduit is perforated at the lower two-thirds portion.

10. The system of claim 1, wherein the at least one conduit is cylindrical and has a diameter between 40 mm and 100 mm.

11. The system of claim 1, further comprising a divider surrounding the layer of mineralized waste.

12. The system of claim 1, further comprising a first separation layer between the conduit and the layer of mineralized waste.

13. The system of claim 12, wherein the first separation layer has a thickness of about 100 mm to about 500 mm.

14. The system of claim 12, further comprising a second separation layer outside of the layer of mineralized waste.

15. The system of claim 14, wherein the first and the second separation layers are composed of materials selected from the group consisting of gravels, tire shreds, and broken glasses.

16. (canceled)

17. (canceled)

18. (canceled)

19. (canceled)

20. The system of claim 12, further comprising a divider between the first separation layer and the layer of mineralized waste.

21. The system of claim 1, further comprising a divider between the conduit and the layer of mineralized waste.

22. (canceled)

23. The system of claim 20, wherein the divider is a porous net.

24. (canceled)

25. A method for oxidizing a material, comprising providing a system comprising at least one conduit configured for air access, and a layer of mineralized waste surrounding the at lease one conduit; and contacting the system with the material to be oxidized.

26. The method of claim 25, wherein the material to be oxidized is solid waste.

27. The method of claim 25, wherein the system for oxidizing methane is placed substantially vertically into the material to be oxidized.

28. The method of claim 25, wherein the system for oxidizing methane is placed slantedly into the material to be oxidized.

29. (canceled)

30. The method of claim 25, wherein the material to be oxidized partially or completely surround the portion of the system that contains mineralized waste.

31. The method of claim 25, further comprising filling additional mineralized waste into the layer of mineralized waste in order to keep the height of the mineralized waste at substantially the same level as the material to be oxidized.

32. The method of claim 25, further comprising adding a nutrient solution into the layer of mineralized waste for every 1-2 m of mineralized waste filled into.

33. The method of claim 25, wherein the system for oxidizing methane further comprises a separation layer between the conduit and the layer of mineralized waste.

34. (canceled)

35. (canceled)

36. (canceled)
